(19) United States
(12) Patent Application Publication
(10) Pub. No.: US 2010/0169118 A1
(43) Pub. Date: Jul. 1, 2010

(54) CENTRALIZED HEALTHCARE DATA MANAGEMENT

(75) Inventors: Margaret E. Rottsolk, Seattle, WA (US); Sean P. Nolan, Bellevua, WA (US)

Correspondence Address: TUROCY & WATSON, LLP, 127 Public Square, 57th Floor, Key Tower, CLEVELAND, OH, 44114, US

(73) Assignee: Microsoft Corporation, Redmond, WA (US)

(21) Appl. No.: 12/345,334

(22) Filed: Dec. 29, 2008

(51) Int. Cl. G06Q 50/00; A61B 5/117; G06Q 30/00; G06F 17/00

(52) U.S. Cl. 705/3; 340/5.82; 600/301; 705/10; 705/14.36

(57) ABSTRACT

A mechanism that allows employers to associate “identity” tokens with a centralized healthcare record is provided. These tokens can be used by wellness applications to target specific behavior related to partnering employers. The innovation also provides for a mechanism that allows employers to review “events” from across the centralized healthcare data ecosystem. These events can be used to track program effectiveness. The events can be fed back into other employer systems such as incentive engines to close the loop with employees.

### BACKGROUND

Recent trends in the healthcare industry have been directed to centralizing storage of healthcare data. This centralization has great benefit to both healthcare entities as well as patients. For instance, transfer of or access to records can be accomplished virtually instantaneously by way of a network connection. Most of these solutions leave the individual patient in control of access to and sharing of their information.

In accordance with centralized storage of healthcare records, individuals are able to make more informed health decisions for themselves and their family. By vesting control of the information in the owner of the information, decisions with regard to sharing or use can be based upon trust, relationship or other parameter. For example, an owner can choose to share their healthcare information with one entity while blocking access by another. Additionally, a user can choose to share healthcare information with an employer while shielding access from a particular service provider (e.g., insurance company).

Because the healthcare information and records are stored in a centralized, network-accessible locations (e.g., Internet), the patient should be able to use their health information wherever and whenever they want. As stated above, it can be possible to share the information, or portion of the information, as desired. In most approaches, access can be regulated by the owner of the information based upon entity, type of information, amount of information, or other desired parameter.

Most recently, centralized healthcare data services are working with doctors, hospitals, employers, pharmacies, insurance providers and manufacturers of health devices (e.g., blood pressure monitors, heart rate monitors) to make it easy to add information electronically to the centralized healthcare record.

With a more complete picture of a family's health, an individual can work with healthcare professionals and with authorized service providers (e.g., Web sites) that connect with healthcare data to make more informed health-related decisions. Unfortunately, traditional approaches have not been designed to integrate effectively with corporations, organization or other groups. Thus, these entities are not able to leverage the powerful benefits of the centralized storage of healthcare data.

### SUMMARY

The following presents a simplified summary of the innovation in order to provide a basic understanding of some aspects of the innovation. This summary is not an extensive overview of the innovation. It is not intended to identify key/critical elements of the innovation or to delineate the scope of the innovation. Its sole purpose is to present some concepts of the innovation in a simplified form as a prelude to the more detailed description that is presented later.

The innovation makes it easy for employers to leverage the open centralized healthcare data ecosystem to engage their members in activities that get or keep them healthy. One motivating factor is the fundamental belief that a healthier base will be less expensive and more productive.

The innovation disclosed and claimed herein, in one aspect thereof, comprises a system that manages group memberships, for example, as they relate to wellness applications and services that employ centralized healthcare data. Effectively, many times a wellness application can offer a particular group a benefit, functionality or preferred status. In accordance with the innovation, this group status can be authenticated as a function of the user identity.

In other aspects, events can be monitored, tracked or otherwise discovered. These events can be used to incentivize a user for an act or penalize for an omission to act. In aspects, the monitoring and tracking of events enables employers to recognize performance based upon a wellness protocol.

As stated above, the innovation essentially enables two scenarios: First, a mechanism that allows employers to associate “identity” tokens with a centralized healthcare record. These tokens can be used by wellness applications to target specific behavior related to partnering employers. Second, a mechanism that allows employers to review “events” from across the centralized healthcare data ecosystem is provided. These events can be used to track program effectiveness. Additionally, the events can be fed back into other employer systems such as incentive engines to close the loop with employees.

In yet another aspect thereof, a machine learning and reasoning component is provided that employs a probabilistic and/or statistical-based analysis to prognose or infer an action that a user desires to be automatically performed.

To the accomplishment of the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

FIG. 1 illustrates an example block diagram of a system that manages identity and event tokens in a centralized healthcare data environment.

FIG. 2 illustrates an example flow chart of procedures that facilitate validating a group identity in accordance with an aspect of the innovation.

FIG. 3 illustrates an example flow chart of procedures that facilitate tracking events in accordance with an aspect of the innovation.

FIG. 4 illustrates an example block diagram of a group identification management component in accordance with an aspect of the innovation.

FIG. 5 illustrates an example block diagram of a group membership determination component within a wellness application in accordance with an aspect of the innovation.

FIG. 6 illustrates an example incentive management component in accordance with an aspect of the innovation.

FIG. 7 illustrates an architecture including a machine learning and reasoning component that can automate functionality in accordance with an aspect of the novel innovation.

FIG. 8 illustrates a block diagram of a computer operable to execute the disclosed architecture.

FIG. 9 illustrates a schematic block diagram of an exemplary computing environment in accordance with the subject innovation.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

As used herein, the term to “infer” or “inference” refer generally to the process of reasoning about or inferring states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources.

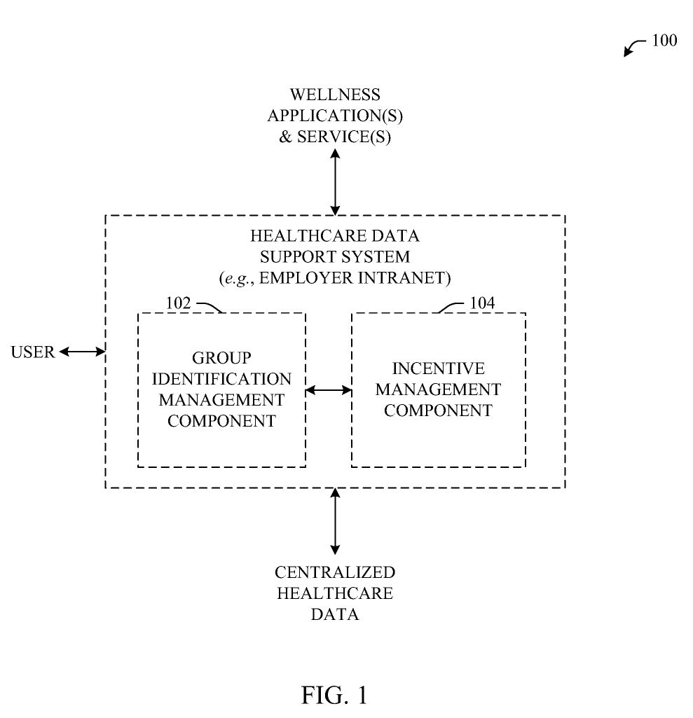

Referring initially to the drawings, FIG. 1 illustrates a system 100 that empowers employers to leverage the open centralized healthcare data ecosystem to engage their members in activities that get or keep them healthy. It is to be understood that one motivation of leveraging the healthcare data ecosystem is the fundamental belief that a healthier employee base will be less expensive and more productive.

Generally, system 100 can include a group identification management component 102 and an incentive management component 104. Together, these sub-components (102, 104) provide valuable features, functions and benefits that enable employers to leverage centralized healthcare data ecosystems.

It will be understood that the centralized healthcare ecosystem can provide a broad and cost-effective palette of choices in terms of partner wellness applications and partners. The innovation provides employers with mechanisms to manage their relationships, track progress and ROI (return on investment). Employers can also be provided with mechanisms to integrate their existing incentives and reporting structures with these partner wellness sites. The innovation disclosed and claimed herein provides for these features, functions and benefits.

The system 100 enables at least two scenarios for employers and the vendors that serve them. As shown in FIG. 1, each of these scenarios is illustrated as a sub-component (102, 104) to the system 100. The group identification management component 102 provides a mechanism that allows employers to associate group “identity” tokens (or other identifier) with a centralized healthcare data record. In aspects, these tokens can be used by wellness applications to target specific behavior related to partnering employers or groups. The incentive management component 104 provides a mechanism that allows employers to review “events” from across the centralized healthcare data ecosystem. As will be described in greater detail below, these events can be used to track program effectiveness. Additionally, the events can input into other employer systems such as incentive engines to close the loop with employees by providing awards, recognition or penalties as appropriate.

It is to be understood and appreciated that employees can opt-in or out at any time with regard to sharing of or access to information. In other words, it is always and entirely up to the individual to both associate employer information with their records and to allow wellness applications they use to “see” what groups they belong to. It will further be appreciated that the cost of not allowing these behaviors may be reduced functionality or higher individual costs as they will not get the benefits of their employer relationships. This tradeoff is exactly in line with how people to perceive their centralized healthcare data experience—employees can choose the trust relationships they are comfortable with in exchange for value.

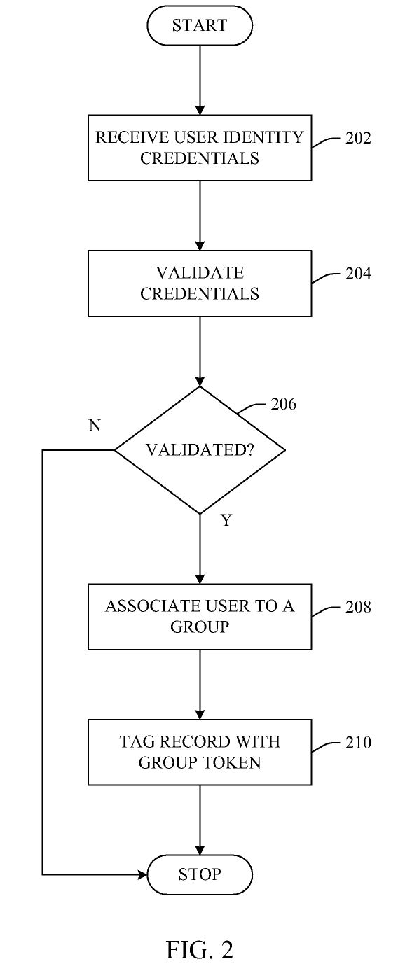

FIG. 2 illustrates an example methodology of associating group identity tokens to centralized healthcare data records in accordance with an aspect of the innovation. While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

At 202, user credentials are received. Here, a user's credentials can be authenticated. In aspects, credentials can range from a username/password combination to a challenge/response to biometric data. While credential examples are given, it is to be understood that most any credentials can be employed to authenticate a user without departing from the spirit and scope of the innovation.

The credentials are validated (e.g., authenticated) at 204. A determination is made at 206 to establish if the user is successfully authenticated. If not validated, as illustrated, the methodology ends. If validated at 206, the user can be associated to a group at 208. Here, the user can identify which group they belong to, for example by way of a user interface. Once associated to a group, records stored within the centralized healthcare data system can be tagged with a particular group token.

In operation, when data is retrieved from the centralized data system, e.g., via a wellness application, the token can be analyzed to establish group membership(s). By way of example, suppose a user accesses a wellness application, here, the data retrieved from the centralized system can be analyzed to determine a user's affiliation with a group. Thus, if applicable, the user can be afforded group benefits (e.g., pricing plan or functionality) as appropriate. Similarly, if data is stored as shown in FIG. 2, the data can be tagged with the group token for later identification and/or classification by an employer.

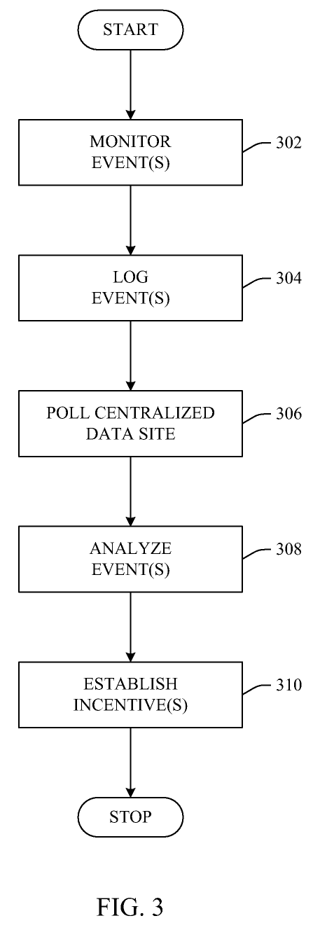

Referring now to FIG. 3, there is illustrated an example methodology of tracking events in accordance with aspects of the innovation. At 302, events can be monitored. For example, an event can be representative of a user taking a blood pressure measurement, heart rate or the like. Each event can be logged at 304. As described herein, the events can be logged into a centralized healthcare data system and tagged with a group token, if appropriate.

As shown in 306, the centralized data site can be polled, for example by an employer. In one aspect, the group tokens can be used to ensure polling and retrieval of data that corresponds to members of the particular group, e.g., employees and their families. Once the data is polled and retrieved, at 308, the event data can be analyzed. Here, the specifics of the event data can be identified.

At 310, incentives can be established based at least in part upon the event data. In operation, an incentive engine can be employed to determine an appropriate incentive (or penalty) based upon the event data. In one aspect, a user can be rewarded for monitoring blood pressure, running on a treadmill or most any other health-related event. Similarly, a user can be penalized for neglecting to perform some designated health-related event.

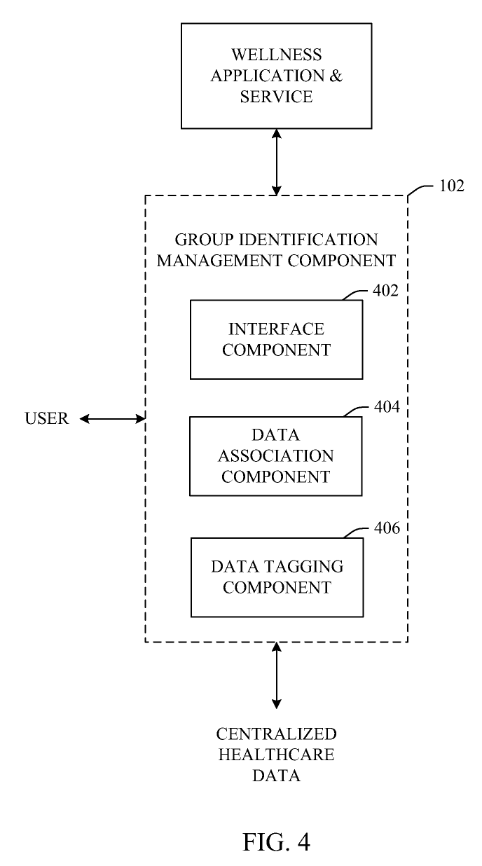

Turning now to FIG. 4, an example group identification management component 102 is shown. As illustrated, the component 102 can include an interface component 402, a group selection component 404 and a data tagging component 406. Together, these sub-components (402, 404, 406) facilitate establishment of group association by way of ‘identity.’

It is to be understood that many of the features, functions and benefits of the innovation as described herein require that the employer enable their employee portals to interact with a centralized healthcare data system. This enablement can be accomplished in a number of manners, including but not limited to, on a custom development basis, by using third party software that is enabled out-of-the-box, or by using an “employer accelerator,” as described below.

As illustrated in FIG. 2, in aspects, the flow begins when the employee logs into their employer portal and signs in as they do regularly. At this point the system knows the identity of the employee, so long as the credentials are validated successfully. On the portal, the employee can be presented with an option to connect their centralized healthcare data records to their employee benefits. This presentation and input can be effected by way of the interface component 402.

Once authorization is granted, the user can employ the interface component 402 to log into the centralized healthcare data system and commence the application authorization for their employer application—which will most often ask for read/write rights on the group “identity” type and at least read rights on the “event” type. It is to be appreciated that additional information can be requested in aspects as appropriate or applicable.

The final connection step is for the employee to associate individual records with the individuals that the employer knows about, for example, which records belong to the employee versus his family, etc. This association can be effected by way of the data association component 404. As illustrated in accordance with the methodology of FIG. 2, the employer application can then write an appropriate “identity” item or token into each linked record. Here, the data tagging component 406 can be employed to write this information into the centralized record(s). Most often, the employer is responsible for the integrity of this item or token, e.g., deleting it when the user is no longer part of the population. Additionally, the employer can store the relationship in their own systems as well.

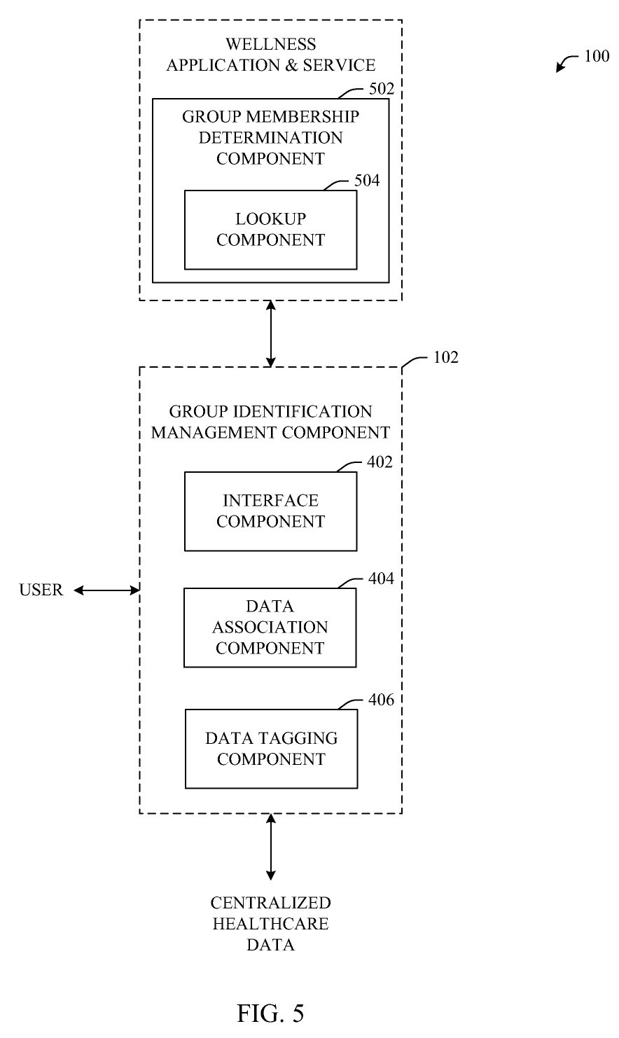

As shown in FIG. 5, a wellness application can include a group membership determination component 502 that is capable of associating a user to a particular group. As described supra, this association can be accomplished by way of the identification token embedded or written into a centralized healthcare data system. A lookup component 504 can be employed to establish group affiliations or memberships based upon the tokens identified.

Continuing with the aforementioned example, once associations are made within the centralized healthcare system, the employee then just goes about their business of using wellness applications. In one aspect, the employee may find the wellness applications through a directory on the employee portal (e.g., intranet), or they may be discovered by the employee directly, for example by searching the Internet.

Some event items are automatically inserted into the record—for example, when the user signs up for a new application. However, other events, for example, when the user attends a class with a nutritionist, can be added directly by event-aware applications (e.g., wellness applications) as the user interacts with them.

Wellness applications that have relationships with employers will most often request read access to the identity type within a centralized healthcare data system. If a user appears at an application with an identity they understand (e.g., by way of the lookup component 504), the application can adjust functionality or benefits—offering new features or automatically granting access to a subscription-only section of the site. Wellness applications can request permission to create event items in a record. These items can represent activity that an employer might like to track and incent on but are not natively understood by the centralized healthcare data infrastructure.

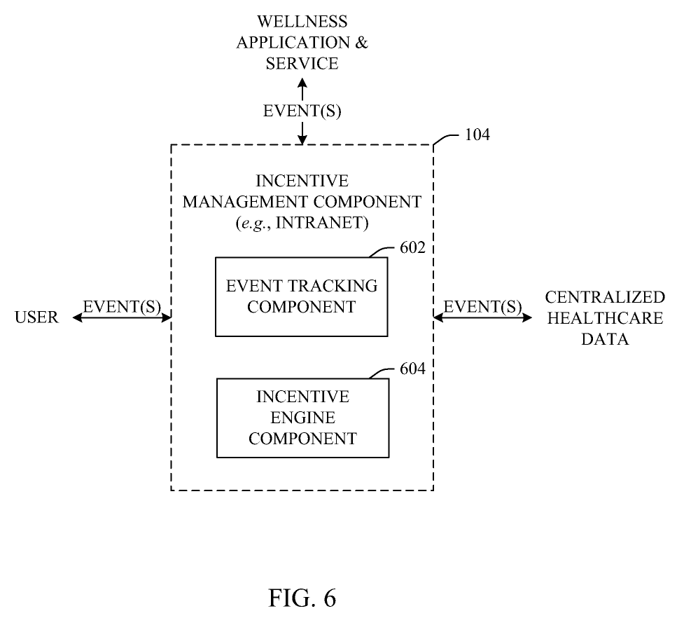

FIG. 6 illustrates an example incentive tracking management component 104 in accordance with aspects of the innovation. Generally, the component 104 can include an event tracking component 602 and an incentive engine component 604. The tracking component 602 is capable of retrieving and analyzing events from the centralized healthcare data sited. In operation, the employer application (e.g., incentive management component 104) can, for example, in offline mode, query (or poll) records for event items that they are interested in. These events can be stored in corporate systems for data mining purposes, and/or funneled into existing incentive engines (604) whereby awards (or penalties) can be granted.

In other aspects, employers may choose to look at non-event activity in the record as well. In operation an employer might want to incent some set of their employees to monitor their blood pressure or blood glucose on a regular basis—not caring what application or device is used to do so. Simply by requesting read access to the appropriate event item types, the employer system (event tracking component 602) can track behavior and offer incentives.

Referring again to a discussion of tracking identity, employer applications assert identity within a healthcare record by creating and maintaining an ‘identity’ item in that record, or group of records. The item may purely represent a group identity, e.g., “employee of ABC Company,” or it may be more granular, e.g., “employee #091506”). If desired, the identity can also include other data such as, but not limited to, demographics such as a validated birth date and gender. In most cases, it is the employer's decision to determine and disclose what information they are putting into their identity tokens.

The below is an example type definition:

```
<complexType name=”IdentityValue”>
    <sequence>
        <!-- recommended vocab: identity-value-codes -->
        <element name=”name” type=”codable-value” />
        <element name=”value” type=”string” />
    </sequence>
</complexType>
<complexType name=”Identity”>
    <sequence>
        <element name=”identity-token” type=”t:IdentityValue”
            minOccurs=”0” maxOccurs=”unbounded” />
        <element name=”expires” type=”dateTime”
            minOccurs=”0” maxOccurs=”1” />
    </sequence>
</complexType>
```

The identity-value-code vocabulary can start with:
Organization (e.g., “ABC Company”)
Organization Subgroup (e.g., “Health Solutions Group” or “Diabetes Cohort”)
Member Number
Full Name
Gender
Birthdate
etc.

It is important to note that identity items are not generally targeted at a particular wellness application. Rather, these items include identifiers that can be used across the full suite of wellness applications that implement identity-based functionality. It is to be understood that, because each identity value type is fully-extensible as a codable-value, employers may create tokens that only one or some wellness applications will understand. Accordingly, employers can decide when they want to enable generalized connections and when they want to do something more one-on-one with a partner.

As described above, the employer is able to maintain their group memberships. In one aspect, an “expires” field defines a way for employers to automatically keep their group memberships up-to-date. Each month (or other designated period) the employer can “refresh” identity tokens for employees that are still present in the system. If an employee leaves the employer, their membership will automatically expire at the end of the period (or some other designated time period).

Turning now to a discussion of tracking events within the system. As described supra, an example event corresponds to some action taken by or an accomplishment of a user. Generally, events come in three varieties:
1. Explicit Common Events are triggered and created by the centralized data system itself. For example, authorizing a record for an application will create an explicit Event item.
2. Explicit Custom Events are triggered and created by wellness applications and correspond to actions or accomplishments that an employer might be interested in but are not understood by the centralized healthcare data system itself.
3. Implicit Events do not actually have a corresponding Event item. Rather, they are computed by an interested employer based on the contents of a healthcare data record. For example, an employer could verify that an employee is taking blood glucose measurements on a regular basis by querying for recent Blood Glucose items.

Equipped just with #1 and #3 above, employers can do quite a bit to create positive incentive programs and understand which wellness applications are interesting and desirable to their employee population. The addition of custom events (#2) makes the set of items that can be tracked and rewarded effectively boundless.

The below is an example type definition:

```
<complexType name=”Event”>
    <sequence>
        <!-- recommended vocab: event-value-codes -->
        <element name=”value” type=”codable-value” />
        <element name=”when” type=”d:date-time” />
        <element name=”extra” type=”string”
        minOccurs=”0” maxOccurs=”1” />
    </sequence>
</complexType>
```

As with Identity tokens, wellness applications can create new event types by extending the “value” codable-value element. This can be done in concert with particular employers or as a general facility multiple employers can use if desired.

It will be appreciated that there can be trust issues for identity and event items. As described above, information in a centralized healthcare record is under the control of the record holder. As a result, it is important that employers and wellness applications, especially those granting awards or access to features, take steps to be sure they are not being “spoofed” with fake data entries.

In most cases, identity or event items need not be digitally signed in order to be trusted. A wellness application that knows it is working with “ABC Company” will need to know ABC Company's application identification (ID) in any case. Verifying that the item was created and updated by this application ID (aka group ID) is most often sufficient to trust the item. It will be appreciated that wellness applications must only trust identity items created by applications they trust.

The same is true for employers looking at event items. Employers must ensure that these items are created and/or updated by the applications they expect. This is the shell application ID for all common events. Implicit events present a different problem. For example, a nefarious employee could just make up blood pressure readings and enter them with the wellness or shell application. An employer application that offers a reward to measure blood pressure on a regular basis could easily be duped into paying out undeserved rewards.

There are at least two ways to approach this issue. In many cases, it may be sufficient to just ignore the problem and absorb fraud as a cost of the program. This of course depends on the reward being offered; as the value increases this becomes less reasonable. If the employer wants a higher degree of trust in the items used to compute implicit events, there are at least a few options. One is to require a digital signature on all items used in the event, and trust only items signed by well-known certificates.

More easily, the employer might simply trust items from a particular application ID. For example, an employer might trust a wellness application controlled by a personal trainer or physician. Finally, an employer may require “validation” in the form of an annotation applied to items by a well-known person or application. It is to be understood that there are many ways to address this issue. Each of the alternative solutions is to be included within the scope of this disclosure and claims appended hereto.

In accordance with the innovation identity and event item types are created. In addition, the platform can be enabled to create event items in a record when certain events occur—to start simply when a record is authorized against an application.

It is important to provide a platform method that allows an employer application to retrieve all of its authorized person/record combinations. Thus, the employer application can keep identity tokens up to date.

Further, aspects of the innovation can be equipped to prevent users from “stealing” functionality from ex-employers. Here, if a user revokes read/write access for identity tokens for an application—either by completely removing authorization or an optional edit—the platform must automatically delete all identity items created by the application that has lost access. It will be understood that this ensures that when the relationship ends, the employee does not retain the identity item that wellness applications use to decide if they should grant additional functionality.

For many employers—especially smaller ones—the innovation can provide an “employer accelerator” application which helps them create their employer application. In operation, among other functionalities, this application can:
Manage the employee signup and auth process;
Manage identity tokens; and
Display simple event reports for employees.

The application can be configurable (or at least customizable) to be able to automatically synchronize identity tokens with an existing employee database or system. Similarly, the accelerator can be able to call customized code when events fire. Thus, the employer can integrate with incentive systems and engines, even if the action is simply “send an email.”

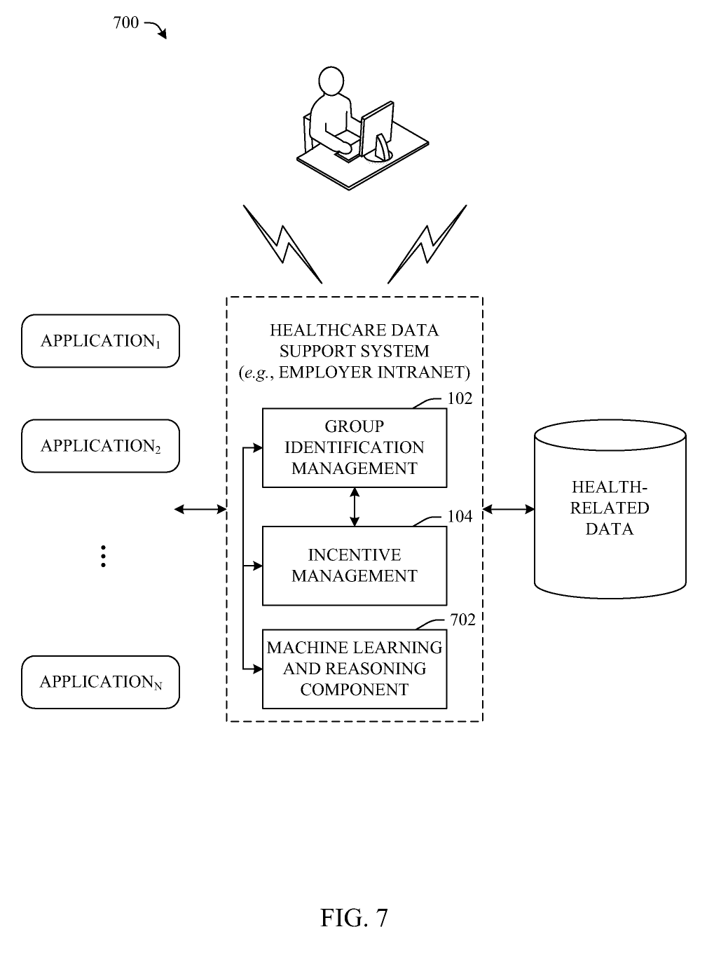

FIG. 7 illustrates a system 700 that employs a machine learning and reasoning component 702 which facilitates automating one or more features in accordance with the subject innovation. The subject innovation (e.g., in connection with granting read/write access) can employ various MLR-based schemes for carrying out various aspects thereof. For example, a process for determining when to grant access, when to revoke access, when to log an event, etc. can be facilitated via an automatic classifier system and process.

A classifier is a function that maps an input attribute vector, x=(x1, x2, x3, x4, xn), to a confidence that the input belongs to a class, that is, f(x)=confidence(class). Such classification can employ a probabilistic and/or statistical-based analysis (e.g., factoring into the analysis utilities and costs) to prognose or infer an action that a user desires to be automatically performed.

A support vector machine (SVM) is an example of a classifier that can be employed. The SVM operates by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

As will be readily appreciated from the subject specification, the subject innovation can employ classifiers that are explicitly trained (e.g., via a generic training data) as well as implicitly trained (e.g., via observing user behavior, receiving extrinsic information). For example, SVM's are configured via a learning or training phase within a classifier constructor and feature selection module. Thus, the classifier(s) can be used to automatically learn and perform a number of functions, including but not limited to determining according to a predetermined criteria when to grant access, when to revoke access, when to write tokens, when to write events, etc.

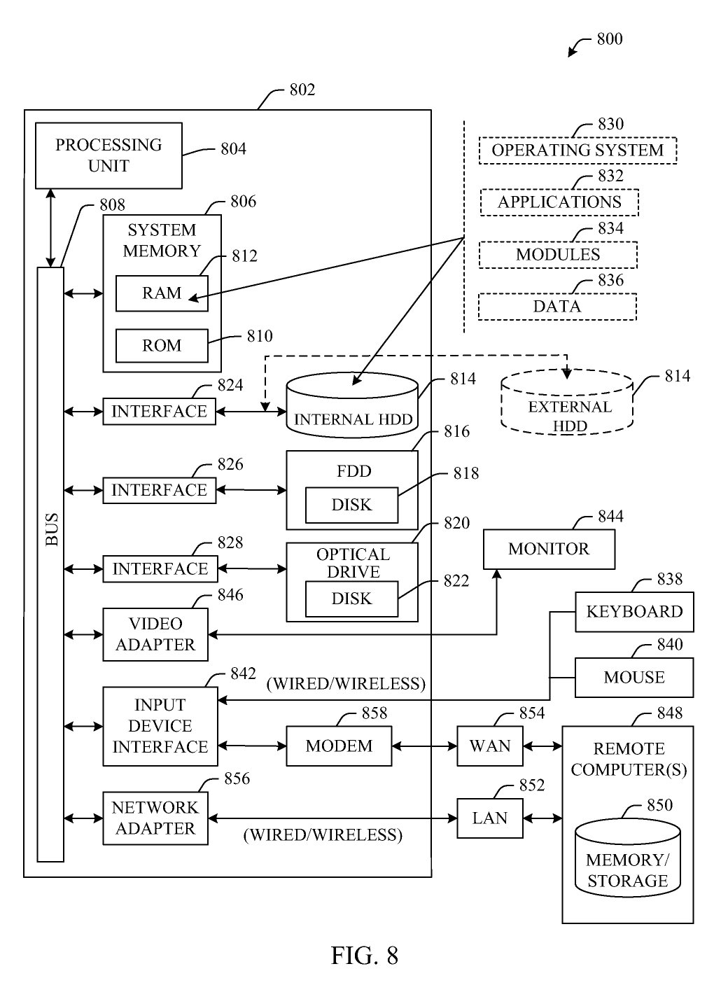

Referring now to FIG. 8, there is illustrated a block diagram of a computer operable to execute the disclosed architecture. In order to provide additional context for various aspects of the subject innovation, FIG. 8 and the following discussion are intended to provide a brief, general description of a suitable computing environment 800 in which the various aspects of the innovation can be implemented. While the innovation has been described above in the general context of computer-executable instructions that may run on one or more computers, those skilled in the art will recognize that the innovation also can be implemented in combination with other program modules and/or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

With reference again to FIG. 8, the exemplary environment 800 for implementing various aspects of the innovation includes a computer 802, the computer 802 including a processing unit 804, a system memory 806 and a system bus 808. The system bus 808 couples system components including, but not limited to, the system memory 806 to the processing unit 804. The processing unit 804 can be any of various commercially available processors. Dual microprocessors and other multi-processor architectures may also be employed as the processing unit 804.

The system bus 808 can be any of several types of bus structure that may further interconnect to a memory bus (with or without a memory controller), a peripheral bus, and a local bus using any of a variety of commercially available bus architectures. The system memory 806 includes read-only memory (ROM) 810 and random access memory (RAM) 812. A basic input/output system (BIOS) is stored in a non-volatile memory 810 such as ROM, EPROM, EEPROM, which BIOS contains the basic routines that help to transfer information between elements within the computer 802, such as during start-up. The RAM 812 can also include a high-speed RAM such as static RAM for caching data.

The computer 802 further includes an internal hard disk drive (HDD) 814 (e.g., EIDE, SATA), which internal hard disk drive 814 may also be configured for external use in a suitable chassis (not shown), a magnetic floppy disk drive (FDD) 816, (e.g., to read from or write to a removable diskette 818) and an optical disk drive 820, (e.g., reading a CD-ROM disk 822 or, to read from or write to other high capacity optical media such as the DVD). The hard disk drive 814, magnetic disk drive 816 and optical disk drive 820 can be connected to the system bus 808 by a hard disk drive interface 824, a magnetic disk drive interface 826 and an optical drive interface 828, respectively. The interface 824 for external drive implementations includes at least one or both of Universal Serial Bus (USB) and IEEE 1394 interface technologies. Other external drive connection technologies are within contemplation of the subject innovation.

The drives and their associated computer-readable media provide nonvolatile storage of data, data structures, computer-executable instructions, and so forth. For the computer 802, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing the methods of the innovation.

A number of program modules can be stored in the drives and RAM 812, including an operating system 830, one or more application programs 832, other program modules 834 and program data 836. All or portions of the operating system, applications, modules, and/or data can also be cached in the RAM 812. It is appreciated that the innovation can be implemented with various commercially available operating systems or combinations of operating systems.

A user can enter commands and information into the computer 802 through one or more wired/wireless input devices, e.g., a keyboard 838 and a pointing device, such as a mouse 840. Other input devices (not shown) may include a microphone, an IR remote control, a joystick, a game pad, a stylus pen, touch screen, or the like. These and other input devices are often connected to the processing unit 804 through an input device interface 842 that is coupled to the system bus 808, but can be connected by other interfaces, such as a parallel port, an IEEE 1394 serial port, a game port, a USB port, an IR interface, etc.

A monitor 844 or other type of display device is also connected to the system bus 808 via an interface, such as a video adapter 846. In addition to the monitor 844, a computer typically includes other peripheral output devices (not shown), such as speakers, printers, etc.

The computer 802 may operate in a networked environment using logical connections via wired and/or wireless communications to one or more remote computers, such as a remote computer(s) 848. The remote computer(s) 848 can be a workstation, a server computer, a router, a personal computer, portable computer, microprocessor-based entertainment appliance, a peer device or other common network node, and typically includes many or all of the elements described relative to the computer 802, although, for purposes of brevity, only a memory/storage device 850 is illustrated. The logical connections depicted include wired/wireless connectivity to a local area network (LAN) 852 and/or larger networks, e.g., a wide area network (WAN) 854. Such LAN and WAN networking environments are commonplace in offices and companies, and facilitate enterprise-wide computer networks, such as intranets, all of which may connect to a global communications network, e.g., the Internet.

When used in a LAN networking environment, the computer 802 is connected to the local network 852 through a wired and/or wireless communication network interface or adapter 856. The adapter 856 may facilitate wired or wireless communication to the LAN 852, which may also include a wireless access point disposed thereon for communicating with the wireless adapter 856.

When used in a WAN networking environment, the computer 802 can include a modem 858, or is connected to a communications server on the WAN 854, or has other means for establishing communications over the WAN 854, such as by way of the Internet. The modem 858, which can be internal or external and a wired or wireless device, is connected to the system bus 808 via the serial port interface 842. In a networked environment, program modules depicted relative to the computer 802, or portions thereof, can be stored in the remote memory/storage device 850. It will be appreciated that the network connections shown are exemplary and other means of establishing a communications link between the computers can be used.

The computer 802 is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, etc.) to provide secure, reliable, fast wireless connectivity. A Wi-Fi network can be used to connect computers to each other, to the Internet, and to wired networks (which use IEEE 802.3 or Ethernet). Wi-Fi networks operate in the unlicensed 2.4 and 5 GHz radio bands, at an 11 Mbps (802.11a) or 54 Mbps (802.11b) data rate, for example, or with products that contain both bands (dual band), so the networks can provide real-world performance similar to the basic 10BaseT wired Ethernet networks used in many offices.

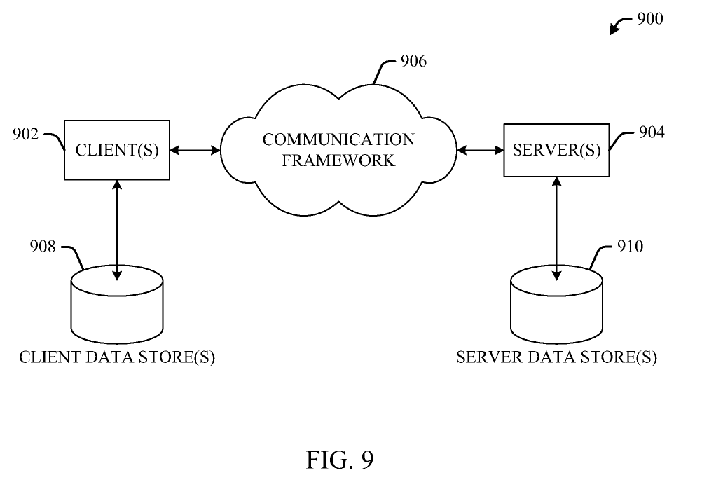

Referring now to FIG. 9, there is illustrated a schematic block diagram of an exemplary computing environment 900 in accordance with the subject innovation. The system 900 includes one or more client(s) 902. The client(s) 902 can be hardware and/or software (e.g., threads, processes, computing devices). The client(s) 902 can house cookie(s) and/or associated contextual information by employing the innovation, for example.

The system 900 also includes one or more server(s) 904. The server(s) 904 can also be hardware and/or software (e.g., threads, processes, computing devices). The servers 904 can house threads to perform transformations by employing the innovation, for example. One possible communication between a client 902 and a server 904 can be in the form of a data packet adapted to be transmitted between two or more computer processes. The data packet may include a cookie and/or associated contextual information, for example. The system 900 includes a communication framework 906 (e.g., a global communication network such as the Internet) that can be employed to facilitate communications between the client(s) 902 and the server(s) 904.

Communications can be facilitated via a wired (including optical fiber) and/or wireless technology. The client(s) 902 are operatively connected to one or more client data store(s) 908 that can be employed to store information local to the client(s) 902 (e.g., cookie(s) and/or associated contextual information). Similarly, the server(s) 904 are operatively connected to one or more server data store(s) 910 that can be employed to store information local to the servers 904.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A system that facilitates use of centralized healthcare data, comprising:
a group identification management component that associates a user as a member of a group, wherein the group qualifies for a predetermined benefit; and
a group membership determination component that validates the user's membership in the group, wherein the validation is based at least in part upon an identity token.

2. The system of claim 1, further comprising an interface component that establishes communication between an employer intranet and a plurality of wellness applications, wherein a subset of the wellness applications offer the predetermined benefit to the group.

3. The system of claim 1, further comprising an interface component that facilitates entry of identity credentials by the user.

4. The system of claim 3, wherein the identity credentials are at least one a user/password combination, challenge/response combination or biometric data.

5. The system of claim 1, further comprising a group selection component that classifies the user into a group based upon identity credentials.

6. The system of claim 5, further comprising a data tagging component that writes the token into a record within the centralized healthcare data, wherein the token represents the membership in the group.

7. The system of claim 6, further comprising a lookup component that recognizes the token in the record and confirms entitlement to the predetermined benefit.

8. The system of claim 1, further comprising an incentive management component that monitors a plurality of health-related events and triggers an award based upon a predetermined policy or rule as a function of a subset of the health-related events.

9. The system of claim 8, further comprising an event tracking component that detects each of the plurality of health-related events.

10. The system of claim 9, further comprising an analysis engine that classifies each of the plurality of health-related events.

11. The system of claim 10, further comprising an incentive engine that triggers the award based at least in part upon the classified health-related events.

12. The system of claim 1, further comprising a machine learning and reasoning component that employs at least one of a probabilistic and a statistical-based analysis that infers an action that a user desires to be automatically performed.

13. A computer-implemented method of leveraging a centralized healthcare data system, comprising:
receiving identity credentials from a user by way of an interface component;
validating the identify;
associating the validated user to a group; and
tagging a user record within the centralized healthcare data system with a token that establishes membership in the group.

14. The computer-implemented method of claim 13, further comprising:
receiving the user record; and
analyzing the token to confirm membership in the group.

15. The computer-implemented method of claim 14, further comprising offering a predetermined benefit based at least in part upon the membership in the group.

16. The computer-implemented method of claim 13, further comprising:
monitoring a plurality of health-related events of the user; and
establishing an incentive based at least upon a subset of the health-related events.

17. A computer-executable system, comprising:
means for monitoring a plurality of health-related events of a user;
means for logging the plurality of health-related events into a centralized healthcare data system;
means for analyzing a subset of the events to establish compliance with a protocol; and
means for rewarding the user based upon a successful compliance with the protocol.

18. The computer-executable system of claim 17, further comprising means for polling the centralized healthcare data system to identify the subset of the events.

19. The computer-executable system of claim 17, further comprising means for associating the user to a group, wherein the group is afforded a membership benefit package.

20. The computer-executable system of claim 19, wherein the membership benefit package is a special pricing plan to access a third party wellness application.
